(19) United States
(12) Patent Application Publication
(10) Pub. No.: US 2011/0196012 A1
(43) Pub. Date: Aug. 11, 2011

(54) DEUTERATED MACROCYCLIC INHIBITORS OF VIRAL NS3 PROTEASE

(75) Inventor: Julie F. Liu, Lexington, MA (US)

(73) Assignee: Concert Pharmaceuticals, Inc., Lexington, MA (US)

(21) Appl. No.: 13/092,801

(22) Filed: Apr. 22, 2011

Related U.S. Application Data
(63) Continuation of application No. PCT/US2009/061766, filed on Oct. 23, 2009.
(60) Provisional application No. 61/107,769, filed on Oct. 23, 2008.

(51) Int. Cl. A61K 31/407; C07D 487/04; C12N 5/071; A61P 31/14

(52) U.S. Cl. 514/411; 540/460; 435/375

(57) ABSTRACT

This invention relates to novel macrocyclic protease inhibitors and their pharmaceutically acceptable salts thereof. This invention also provides compositions comprising at least one compound of this invention and the use of such compositions in methods of treating a flavivirus infection or liver fibrosis in a patient in need thereof.

### RELATED APPLICATIONS

This application is a continuation of PCT International Application No. PCT/US09/61766, filed on Oct. 23, 2009, pending, which claims the benefit of U.S. Provisional Application Ser. No. 61/107,769, filed Oct. 23, 2008. The disclosures of the foregoing applications are hereby incorporated by reference herein in its entirety.

### TECHNICAL FIELD

This invention relates to novel macrocyclic protease inhibitors and pharmaceutically acceptable salts thereof. This invention also provides compositions comprising one or more compounds of this invention and the use of such compositions in methods of treating a flavivirus infection or liver fibrosis in a patient in need thereof.

### BACKGROUND

ITMN-191, also known as 4-fluoro-2,3-dihydro-1H-isoindole-2-carboxylic acid (2R,6S,12Z,13aS,14aR,16aS)-6-(tert-butoxycarbonylamino)-14a-[N-(cyclopropylsulfonyl)carbamoyl]-5,16-dioxo-1,2,3,5,6,7,8,9,10,11,13a,14,14a,15,16,16a-hexadecahydrocyclopropa[e]pyrrolo[1,2-a][1,4]diazacyclopentadecin-2-yl ester, is a macrocyclic molecule that inhibits hepatitis C virus (HCV) NS3 protease.

ITMN-191 is currently in clinical trials for the treatment of HCV infection as a monotherapy and in combination with pegylated interferon alpha-2a and ribavirin.

Despite the potential beneficial activities of ITMN-191, there is a continuing need for new compounds to treat the aforementioned diseases and conditions.

### DETAILED DESCRIPTION OF ILLUSTRATIVE EMBODIMENTS

### Definitions

The terms “ameliorate” and “treat” are used interchangeably and include both therapeutic and prophylactic treatment. Both terms mean decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, for example, a preparation of ITMN-191 will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada E et al., Seikagaku 1994, 66:15; Gannes L Z et al., Comp Biochem Physiol Mol Integr Physiol 1998, 119:725.

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopologue” refers to a species that differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium. “Stereoisomer” refers to both enantiomers and diastereomers. “Tert”, “^{t}”, and “t-” each refer to tertiary. “US” refers to the United States of America.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides compounds of Formula I:

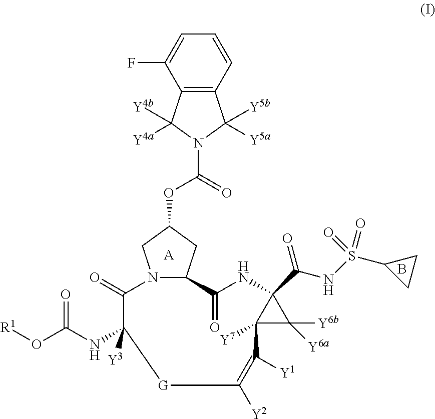

and the pharmaceutically acceptable salts thereof, a wherein:
Ring A contains 0 to 6 deuterium atoms;
Ring B contains 0 to 5 deuterium atoms;
R¹ is a t-butyl group containing 0 to 9 deuterium atoms;
G is an n-pentylene group containing 0 to 10 deuterium atoms;
each Y is independently hydrogen or deuterium;
with the proviso that when Ring A contains 0 deuterium atoms and R¹ and G each contain 0 deuterium atoms, then Ring B contains 1-5 deuterium atoms.

An “n-pentylene” group having 0 deuterium atoms has the formula —(CH₂)₅—. A “t-butyl” group containing 0 deuterium atoms has the formula —C(CH₃)₃. According to the present invention any hydrogen in either the t-butyl group that is R¹, or the n-pentylene group that is G can be replaced with deuterium.

In one embodiment, ring A contains 0 or 6 deuterium atoms.

In another embodiment, ring B contains 0 or 5 deuterium atoms.

In another embodiment, each Y⁴ is the same.

In another embodiment, each Y⁵ is the same. In one aspect of this embodiment, each of Y^{4a}, Y^{4b}, Y^{5a} and Y^{5b} are the same.

In another embodiment, each Y⁶ is the same. In one aspect of this embodiment, each Y⁶ is hydrogen.

In another embodiment, Y¹ and Y⁷ are the same. In one aspect of this embodiment, Y¹ and Y⁷ are hydrogen.

In another embodiment of Formula I, R¹ is selected from —C(CH₃)₃ and —C(CD₃)₃.

In still another embodiment, each carbon atom in G is independently bound to two hydrogen atoms or two deuterium atoms. In one aspect of this embodiment, G is selected from —(CH₂)₅— and —(CD₂)₅—. In another aspect of this embodiment G is selected from —(CH₂)₅— and —(CD₂)₅—; each of Y^{4a}, Y^{4b}, Y^{5a} and Y^{5b} are the same; Y¹, Y^{6a}, Y^{6b} and Y⁷ are hydrogen; ring A contains 0 or 6 deuterium atoms; ring B contains 0 or 5 deuterium atoms; and R¹ is selected from —C(CH₃)₃ and —C(CD₃)₃.

In yet another embodiment, the compound is selected from any one of the compounds set forth below:

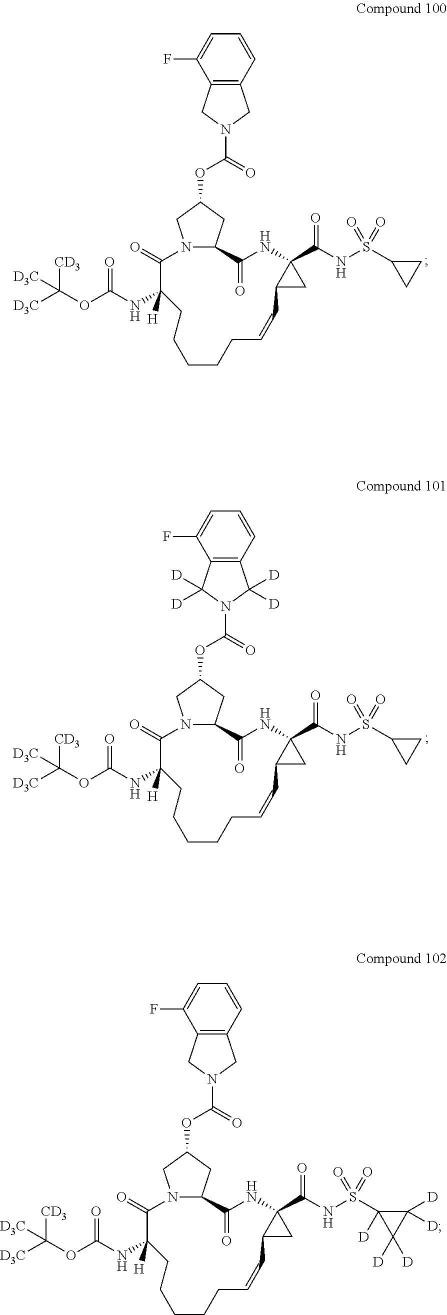

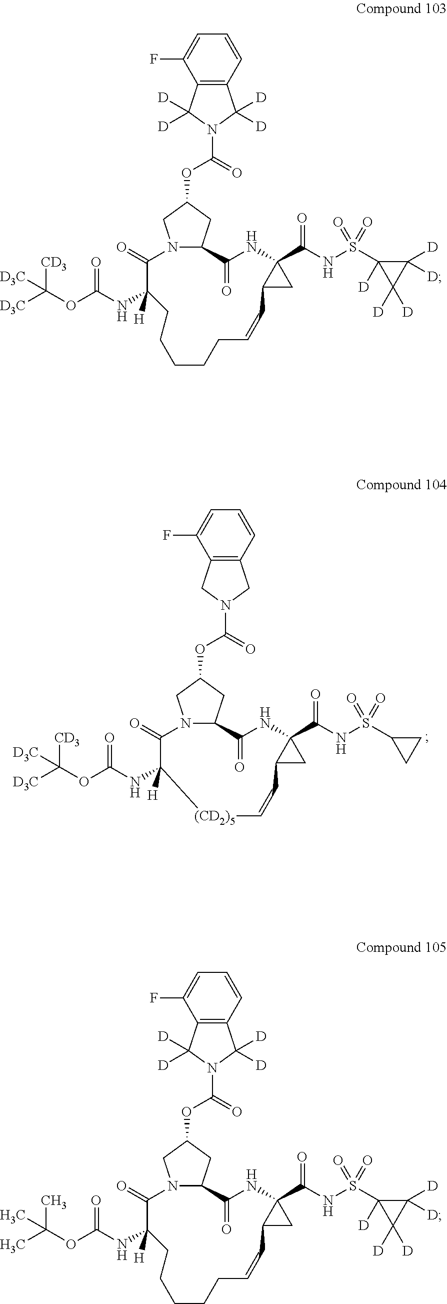

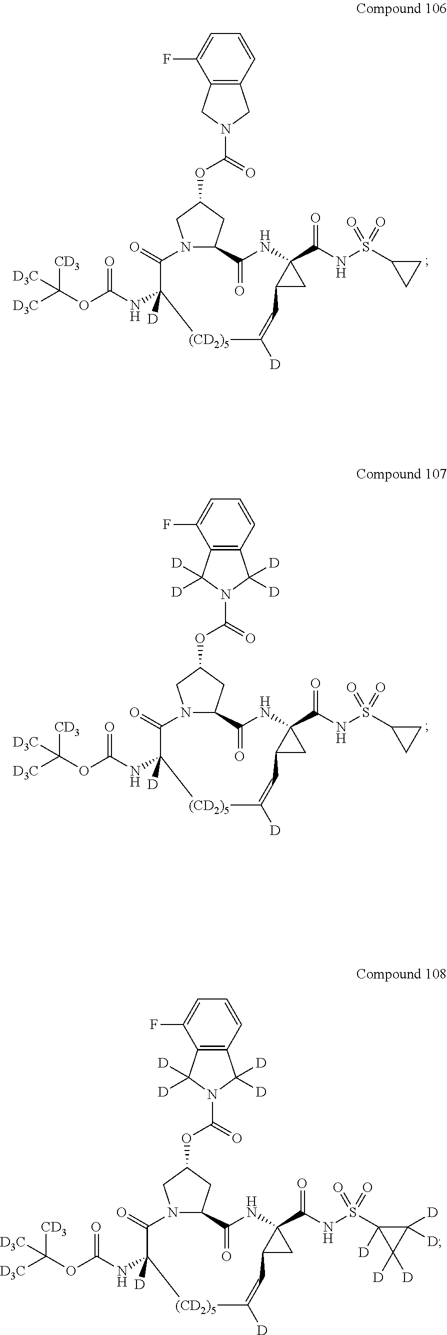

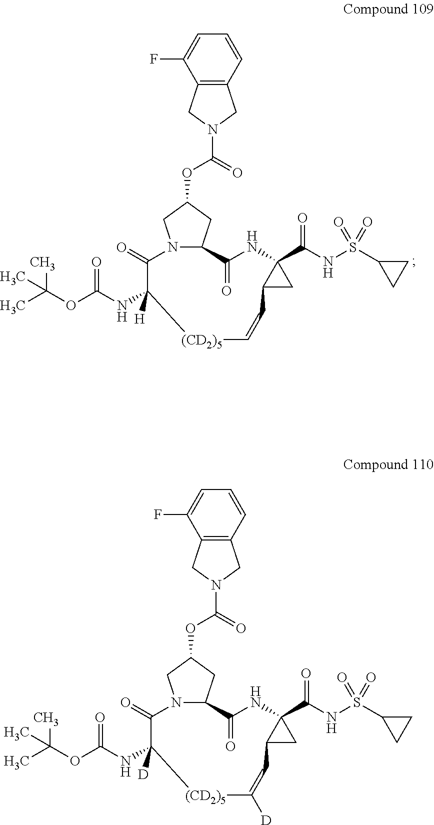

or a pharmaceutically acceptable salt of any of the foregoing.

In another embodiment, an example of a compound of Formula I is Compound 111 below:

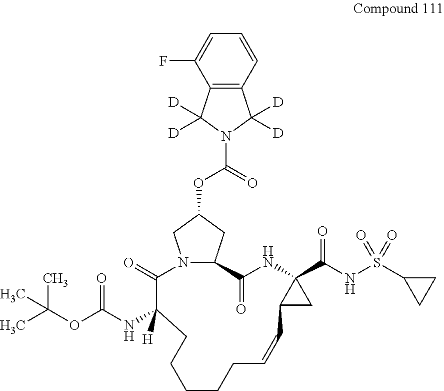

or a pharmaceutically acceptable salt thereof.

In another set of embodiments, any atom not designated as deuterium in any of the embodiments set forth above is present at its natural isotopic abundance.

The synthesis of compounds of Formula I can be readily achieved by synthetic chemists of ordinary skill by reference to the Exemplary Synthesis and Examples disclosed herein. Relevant procedures and intermediates are disclosed, for instance, in PCT publication WO2005037214 and US Published application US20050267018.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

### Exemplary Syntheses

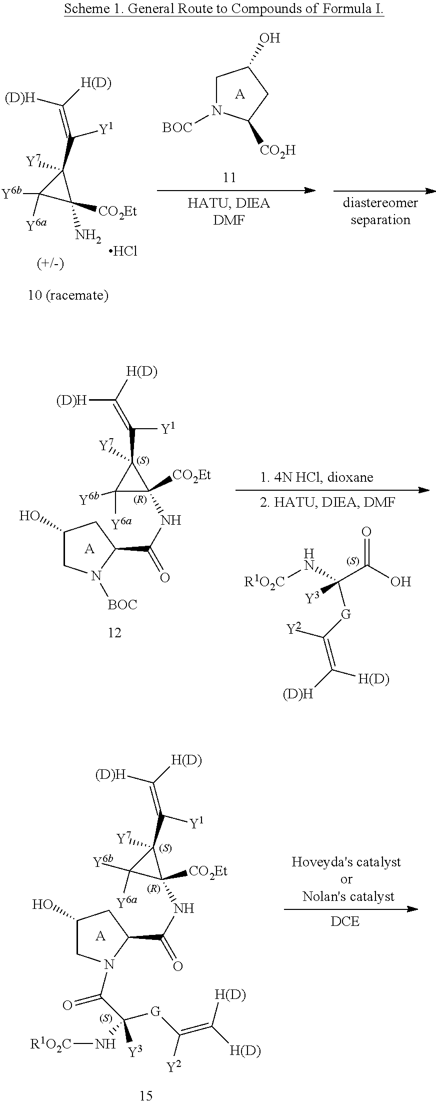

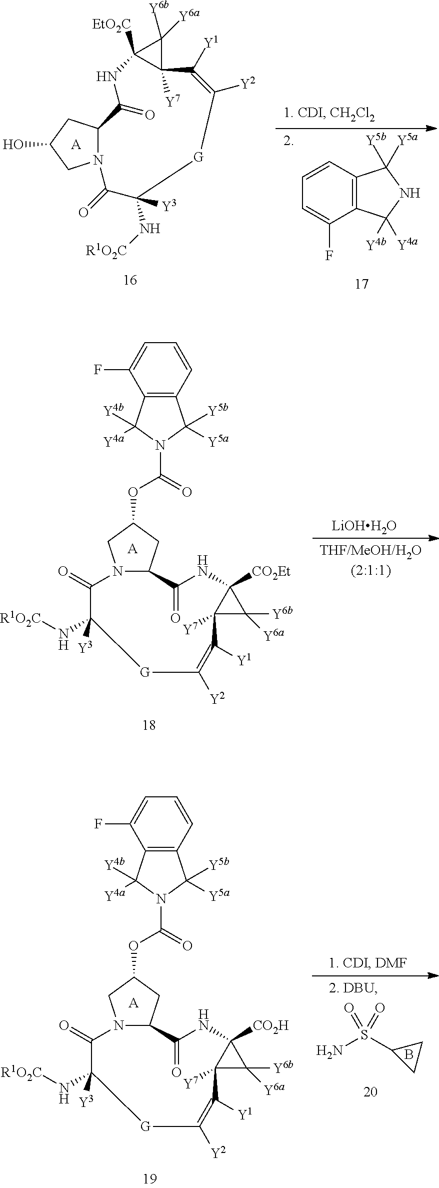

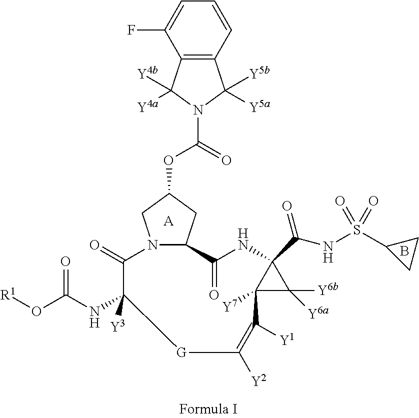

A convenient method for synthesizing compounds of Formula I is depicted in Scheme 1 and follows the general methods of WO2005037214 and US20050267018. Thus, racemic appropriately-deuterated 10 is coupled with hydroxy-L-proline derivative in the presence of O-(7-azabenzotriazol-1-yl)-N,N,N′,N′-tetramethyluronium hexafluorophosphate (“HATU”) 11 in the presence of diisopropylethyl amine (DIEA) to afford a diastereomeric mixture, which may be separated by chromatography to produce 12. Alternatively, appropriately-deuterated enantiopure (1R,2S)-10 may be coupled with 11 to afford 12 directly. Compound 12 is deprotected with HCl and coupled to carboxylic acid 14 to provide compound 15. Metathesis cyclization in dichloroethane (DCE) or a similar solvent using a suitable catalyst, such as “Hoveyda's catalyst” (CAS No. 205815-80-1) or “Nolan's catalyst” (CAS No. 223415-64-3), affords compound 16. Treatment of compound 16 with carbonyldiimidazole (CDI), and then amine 17 in the presence of 1,8-Diazabicyclo[5.4.0]undec-7-ene (DBU) yields compound 18. Hydrolysis of the methyl ester moiety in compound 18 with lithium hydroxide affords compound 19. Treatment with CDI, followed by DBU and sulfonamide 20 provides compounds of Formula I.

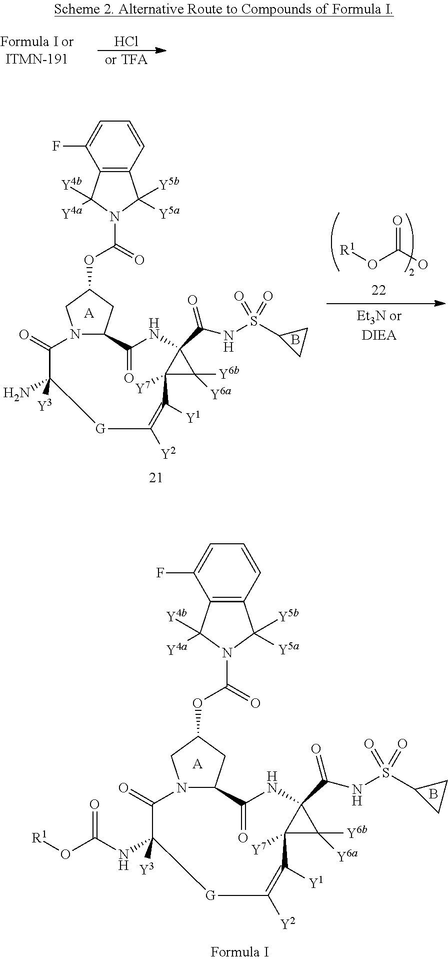

Existing compounds of Formula I (such as made via Scheme 1) or ITMN-191 may be used as starting material to make other compounds of Formula I, as depicted in Scheme 2. Thus, compounds of Formula I or ITMN-191 may be treated with acid, for example HCl or trifluoroacetic acid (TFA), to afford amine 21. Treatment of 21 with appropriately-deuterated dicarbonate 22 provides compounds of Formula I.

An appropriately deuterated dicarbonate 22 includes, for example, known di-tert-butyl-d₁₈ dicarbonate (see Lin, E K et al., Proceedings of SPIE—The International Society for Optical Engineering, 2002, 4690 (Pt. 1, Advances in Resist Technology and Processing XIX): 313-320; or prepared from commercially-available tert-butanol-d₁₀ following the general procedures of European Patent publication EP468404 and of Werstiuk, N H, et al., Can J Chem, 1985, 63 (2): 526-9) may be used to provide compounds of Formula I wherein R¹ is —C(CD₃)₃.

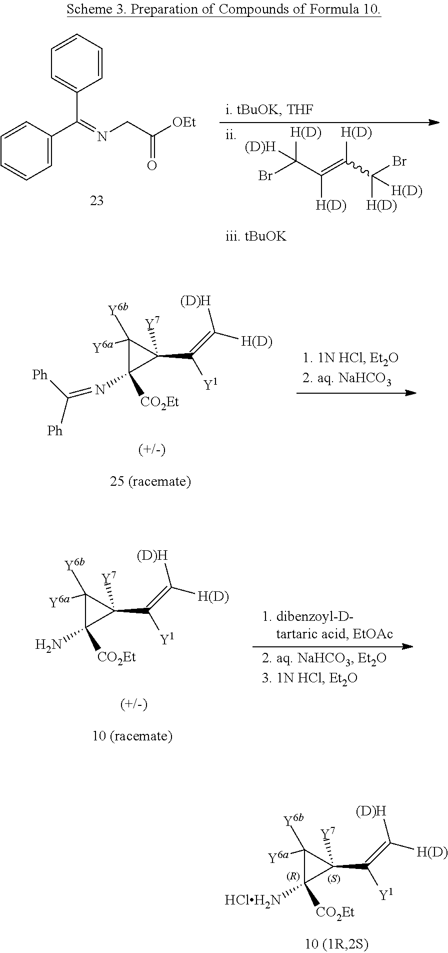

Scheme 3 depicts the preparation of an appropriately deuterated compound 10 in either racemic or enantiopure form. Following the general methods of U.S. Pat. No. 6,268,207 and those of Rancourt, J et al., J Med Chem, 2004, 47:2511-2522, commercially-available N-(diphenylmethylene)glycine ethyl ester 23 is treated sequentially with potassium tert-butoxide, appropriately-deuterated dibromide 24, and potassium tert-butoxide (tBuOK) to provide racemic 25. Treatment with aqueous HCl followed by aqueous sodium bicarbonate affords racemic 10. Enantiomeric resolution via crystallization as the dibenzoyl-D-tartaric acid salt, followed by generation of the HCl salt, affords single enantiomer (1R,2S)-10.

An appropriately deuterated dibromide 24 includes, for example, (a) known dibromide 1,4-dibromo-2-(E)-butene-1,1,2,3,4,4-d₆

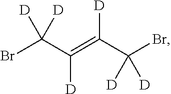

which may be used in the synthesis of compounds of Formula I wherein Y¹, Y^{6a}, Y^{6b}, and Y⁷ are deuterium; and (b) known dibromide 1,4-dibromo-2-butene-2,3-d₂

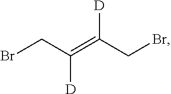

which may be used in the synthesis of compounds of Formula I wherein Y¹ and Y⁷ are deuterium.

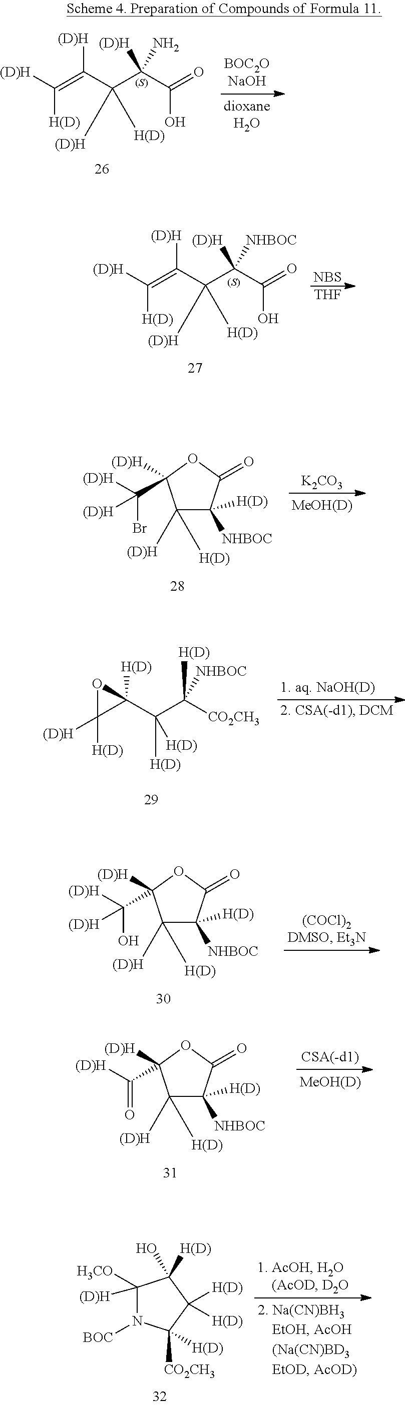

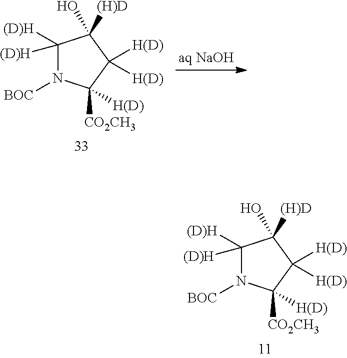

Scheme 4 depicts the preparation of an appropriately deuterated compound 11. Appropriately-deuterated (S)-allylglycine 26 is BOC-protected to afford 27 according to the general methods of Kaul, R et al., J Org Chem, 2005, 70 (10):3838-3844. Following the general methods of Kurokawa, N et al., JACS, 1986, 108:6041-6043, and substituting deuterated reagents and solvents where appropriate, 27 is treated with NBS to afford lactone 28, which is treated with K₂CO₃ in methanol or methanol-d₁ to provide epoxide 29. Treatment with NaOH or NaOD, followed by camphorsulfonic acid or camphorsulfonic acid-d₁ (prepared from commercially-available (1S)-(+)-10-camphorsulfonyl chloride and commercially-available NaOD) provides lactone 30. Swern oxidation affords aldehyde 31, which is treated with camphorsulfonic acid in methanol or camphorsulfonic acid-d₁ (CSA-d₁) in methanol-d₁ to provide compound 32. Treatment of 32 with acetic acid in water, or acetic acid-d₁ (AcOD) in D₂O, followed by reduction with sodium cyanoborohydride in ethanol/acetic acid or sodium cyanoborodeuteride in ethanol-d₁ and acetic acid-d₁ provides compound 33. Hydrolysis with aqueous NaOH affords compound 11.

An appropriately-deuterated (S)-allylglycine 26 includes, for example, (S)-allylglycine-d₆

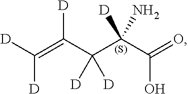

which may be prepared according to the methods of Rees, DO et al., J Label Comp Radiopharm, 2007, 50 (5-6):399-401 from known allyl-d₅-iodide (see Nandi, S et al., J Phys Chem A, 2001, 105 (32):7514-7524).

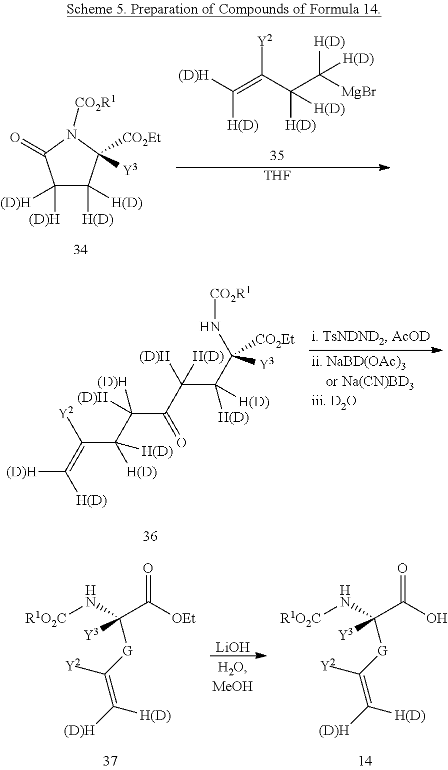

Scheme 5 depicts the preparation of appropriately-deuterated compound 14. Following the general methods of Miao, W et al., Lett Org Chem, 2006, 3 (6):489-491, appropriately-deuterated 34 is treated with appropriately-deuterated Grignard reagent 35 to provide ketone 36. Sequential treatment with commercially-available p-toluenesulfonylhydrazide-N,N,N-d₃ in AcOD, followed by either known sodium triacetoxyborodeuteride (see Robins, M J et al., Tetrahedron, 1997, 53 (2):447-456) or commercially-available sodium cyanoborodeuteride, followed by D₂O affords compound 37. Ester hydrolysis with LiOH in aqueous methanol yields compound 14.

An appropriately deuterated compound 34 may be prepared from deuterated versions of L-glutamic acid. In one example, commercially-available L-glutamic-2,3,3,4,4-d₅ acid is cyclized in D₂O, esterified via treatment with SOCl₂ and EtOD, and acylated via treatment with DMAP and either BOC₂O or known di-tert-butyl-d₁₈ dicarbonate (see Lin, E K et al., Proceedings of SPIE—The International Society for Optical Engineering, 2002, 4690 (Pt 1, Advances in Resist Technology and Processing XIX):313-320) following the general methods of Cappon, J J et al., Recueil des Travaux Chimiques des Pays-Bas, 1992, 111 (12):517-23; and Harris, P W R et al., Org Biomol Chem, 2006, 4 (14):2696-2709.

An appropriately deuterated compound 35 may be prepared from deuterated versions of 1,4-dibromobutane. For example, following the general methods of Hoye, T R et al., Syn Comm, 2001, 31 (9):1367-1371; and of Kraus, G A et al., Synthesis, 1984, 10:885, treatment of commercially-available 1,4-dibromobutane-d₈ with HMPA affords 4-bromo-1-butene-d₇. Treatment of this bromide with magnesium in THF according to the general methods of de Meijere, A et al., Org Synth, 2005, 81:14-25 affords a version of Grignard reagent 35 containing seven deuterium atoms.

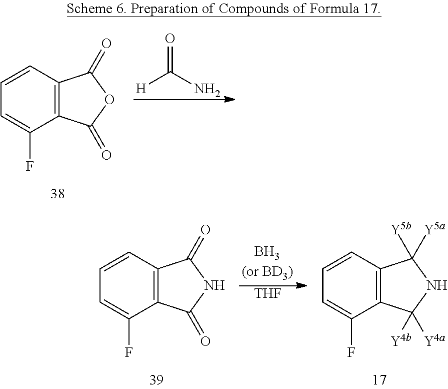

Scheme 6 depicts the preparation of an appropriately deuterated compound 17. Following the general methods of WO2005037214, commercially-available 3-fluorophthalic anhydride 38 is heated with formamide to provide compound 39. Reduction with either borane or borane-d₃ provides appropriately-deuterated amine 17.

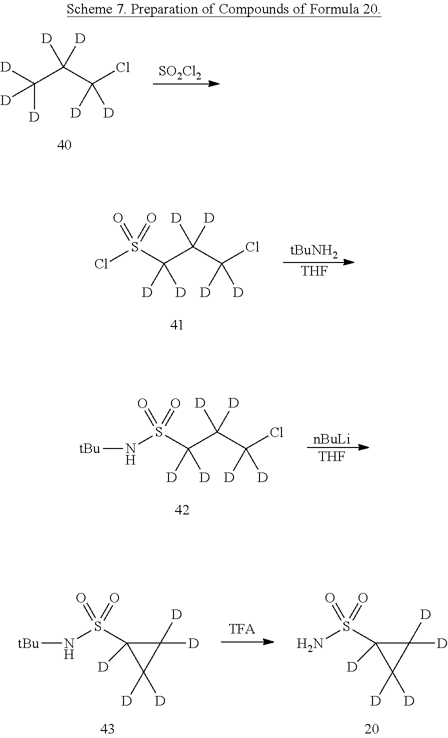

Scheme 7 depicts the preparation of an appropriately deuterated compound 20. Following the general methods of Helwig, D et al., Journal für Praktische Chemie (Leipzig), 1980, 322 (2):281-90, commercially-available 1-chloropropane-d₇ (40) is treated with SO₂Cl₂ to provide sulfonyl chloride derivative 41. Treatment with t-butylamine affords compound 42. Cyclization via treatment with nBuLi provides compound 43, and deprotection with TFA affords compound 20. One skilled in the art will appreciate that other examples of compound 40 bearing different levels of deuteration may be used in Scheme 7 to provide alternate examples of compound 20.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of Formula I and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pharmaceutical compositions (preferably, pyrogen-free pharmaceutical compositions) comprising an effective amount of at least one compound of Formula I (e.g., including any of the formulae herein), or a pharmaceutically acceptable salt thereof; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

In another embodiment, a composition of this invention further comprises a second therapeutic agent. The second therapeutic agent may be selected from any compound or therapeutic agent known to have or that demonstrates advantageous properties when administered with a compound having the same mechanism of action as ITMN-191. Such agents include those indicated as being useful in combination with ITMN-191, including but not limited to, those described in United States patent publication Nos. US 2005267018 and US 2007054842, and in PCT publication No. WO 2005037214.

Preferably, the second therapeutic agent is an agent useful in the treatment or prevention of a disease or condition selected from flavivirus infections and liver fibrosis.

In one embodiment, the second therapeutic agent is selected from interferon-alpha and derivatives thereof (including synthetic IFN-a, pegylated IFN-a, glycosylated IFN-a, consensus IFN-a, and analogs of naturally occurring or synthetic IFN-a), interferon-beta, interferon tau, interferon omega, interferon gamma, IL-28b and active polypeptide portions thereof, IL-28a and active polypeptide portions thereof, IL-29 and active polypeptide portions thereof, other Type 1 interferon receptor agonists, other Type II receptor agonists, other Type III interferon receptor agonists, pirfenidone or a pirfenidone analog, thymosin-a, ribavirin, levovirin, viramidine, a nucleoside analog, a TNF antagonist, an NS5B inhibitor, an IMPDH inhibitor, a ribozyme, antisense or siRNA antiviral agent targeted against a flavivirus, or another antiviral agent.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat (therapeutically or prophylactically) the target disorder. For example, to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., Cancer Chemother. Rep, 1966, 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from about 0.01 mg to about 100 mg/kg patient body weight per day in 1 to 5 divided doses per day. In some embodiments, an effective amount of a compound of this invention can range from about 0.5 mg to about 75 mg/kg patient body weight per day in 1 to 5 divided doses per day.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for Compound 1.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides methods of modulating the activity of viral NS3 protease, comprising contacting a cell infected with a flavivirus with one or more compounds of Formula I herein or a pharmaceutically acceptable salt thereof.

According to another embodiment, the invention provides a method of treating a disease that is beneficially treated by ITMN-191 in a subject, comprising the step of administering to the subject an effective amount of a compound or a composition of this invention. Such diseases include, but are not limited to, flavivirus infections and liver fibrosis.

In one particular embodiment, the method of this invention is used to treat an HCV infection in a subject in need thereof.

In another embodiment, any of the above methods of treatment comprises the further step of co-administering to the subject one or more second therapeutic agents. The choice of second therapeutic agent may be made from any second therapeutic agent known to be useful for co-administration with ITMN-191. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

In one aspect of the foregoing embodiments, the subject is a patient in need of the treatment.

In particular, the invention provides a method of treating flavivirus infection or liver fibrosis (including liver fibrosis resulting from an HCV infection) by co-administering to a patient in need thereof: a) a pharmaceutical composition comprising a compound of Formula I and a pharmaceutically acceptable carrier; and b) a second therapeutic agent selected from one or more of: interferon-alpha and derivatives thereof (including synthetic IFN-a, pegylated IFN-a, glycosylated IFN-a, consensus IFN-a, and analogs of naturally occurring or synthetic IFN-a), interferon-beta, interferon tau, interferon omega, interferon gamma, IL-28b and active polypeptide portions thereof, IL-28a and active polypeptide portions thereof, IL-29 and active polypeptide portions thereof, other Type 1 interferon receptor agonists, other Type II receptor agonists, other Type III interferon receptor agonists, pirfenidone or a pirfenidone analog, thymosin-a, ribavirin, levovirin, viramidine, a nucleoside analog, a TNF antagonist, an NS5B inhibitor, an IMPDH inhibitor, a ribozyme, antisense or siRNA antiviral agent targeted against a flavivirus, or another antiviral agent In some embodiments, the combination therapy set forth above is used to treat an HCV infection in to a patient in need thereof.

In a more specific embodiment the invention provides a method for treating an HCV infection in a patient in need thereof comprising the step of co-administering to the patient: a) a pharmaceutical composition comprising a compound of Formula I and a pharmaceutically acceptable carrier; b) pegylated interferon alpha; and c) ribavirin.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I alone or together with one or more of the above-described second therapeutic agents in the manufacture of a medicament, either as a single composition or as separate dosage forms, for treatment or prevention in a patient of a disease, disorder or symptom set forth above. Another aspect of the invention is a compound of Formula I for use in the treatment or prevention in a patient of a disease, disorder or symptom thereof delineated herein.

### Diagnostic Methods and Kits

The compounds and compositions of this invention are also useful as reagents in methods for determining the concentration of ITMN-191 in solution or biological sample such as plasma, examining the metabolism of ITMN-191 and other analytical studies.

According to one embodiment, the invention provides a method of determining the concentration, in a solution or a biological sample, of ITMN-191, comprising the steps of:

a) adding a known concentration of a compound of Formula Ito the solution of biological sample;

b) subjecting the solution or biological sample to a measuring device that distinguishes ITMN-191 from a compound of Formula I;

c) calibrating the measuring device to correlate the detected quantity of the compound of Formula I with the known concentration of the compound of Formula I added to the biological sample or solution; and

d) measuring the quantity of ITMN-191 in the biological sample with said calibrated measuring device; and

e) determining the concentration of ITMN-191 in the solution of sample using the correlation between detected quantity and concentration obtained for a compound of Formula I.

Measuring devices that can distinguish ITMN-191 from the corresponding compound of Formula I include any measuring device that can distinguish between two compounds that differ from one another only in isotopic abundance. Exemplary measuring devices include a mass spectrometer, NMR spectrometer, or IR spectrometer.

In another embodiment, the invention provides a method of evaluating the metabolic stability of a compound of Formula I comprising the steps of contacting the compound of Formula I with a metabolizing enzyme source for a period of time and comparing the amount of the compound of Formula I with the metabolic products of the compound of Formula I after the period of time.

In a related embodiment, the invention provides a method of evaluating the metabolic stability of a compound of Formula I in a patient following administration of the compound of Formula I. This method comprises the steps of obtaining a serum, urine or feces sample from the patient at a period of time following the administration of the compound of Formula I to the subject; and comparing the amount of the compound of Formula I with the metabolic products of the compound of Formula I in the serum, urine or feces sample.

The present invention also provides kits for use to treat an HCV infection. These kits comprise (a) a pharmaceutical composition comprising a compound of Formula I or a salt thereof, wherein said pharmaceutical composition is in a container; and (b) instructions describing a method of using the pharmaceutical composition to treat an HCV infection.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In an embodiment of the kits of this invention, the composition comprising the second active agent may be in a vessel or container that is separate from the vessel containing the composition comprising a compound of Formula I.

### EXAMPLES

### Example 1

### Synthesis of 1,1,3,3-d₄-4-Fluoroisoindoline (17)

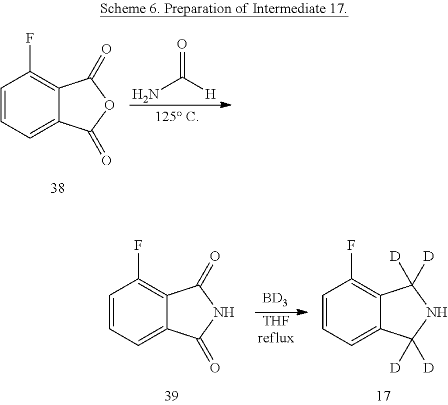

Step 1. 3-Fluorophthalimide (39). A solution of 3-fluorophthalic anhydride 38 (1.00 g, 6.02 mmol) in formamide (12.0 mL) was stirred at 125° C. for 2 hours. The reaction mixture was then cooled to room temperature and water (36.0 mL) was added. The mixture was stirred at room temperature until a white precipitate formed at which time the solids were filtered, washed with water, and dried in-vacuo to afford 39 (0.364 g, 37% yield) as a white solid. MS (M+H): 166.1

Step 2. 1,1,3,3-d₄-4-Fluoroisoindoline. A 1M solution of BD₃ in THF (40.0 mL, 40.0 mmol, Cambridge Isotope Laboratories, 98% D) was added dropwise to a solution of 39 (1.65 g, 10.0 mmol) in THF (2.00 mL). The reaction mixture was stirred at reflux for 18 hours then was cooled to 0° C. Methanol (1.62 mL, 40 mmol) was added dropwise and the mixture was allowed to warm to room temperature. The mixture was then acidified with 6M HCl and stirred at reflux for 1 hour. The resulting mixture was cooled to room temperature, concentrated to remove organic solvents, and diluted with water. The aqueous solution was extracted with diethyl ether (2×50 mL) and CH₂Cl₂ (2×50 mL) then brought to pH 11 by addition of 3M NaOH. The aqueous layer was then diluted with water and extracted with diethyl ether (3×100 mL). The combined organic extracts were dried (Na₂SO₄), filtered and concentrated to afford 17 (376 mg, 27% yield) as a light brown solid. MS (M+H): 142.1.

### Example 2

### Synthesis of (tert-Butyl-d₉) 1,2,2,2-Tetrachloroethyl carbonate (22a)

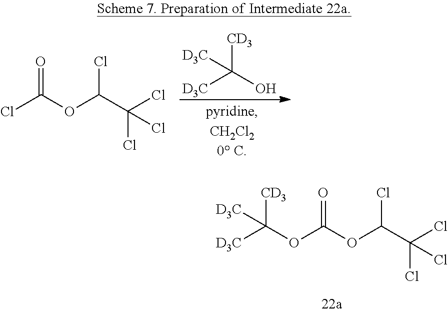

Tert-Butyl-d₉1,2,2,2-Tetrachloroethyl carbonate d₉ (22a). To a solution of tert-butanol-d₉ (915 μL, 8.53 mmol, CDN, 99% D) in CH₂Cl₂ (8.00 mL) at 0° C. was added 1,2,2,2-tetrachloroethyl chloroformate (2.00 g, 8.12 mmol) followed by pyridine (722 μL, 8.93 mmol). The reaction was then stirred at 0° C. for 4 hours and was then filtered through Celite to remove the precipitated pyridine-HCl salts. The filter cake was then rinsed with additional CH₂Cl₂ and the combined CH₂Cl₂ solution was then washed with water, dried (MgSO₄), filtered, concentrated, and dried in-vacuo to afford 22a as a white solid (2.11 g, 89% yield) which was used without purification.

### Example 3

### Synthesis of (2R,6S,13aS,14aR,16aS,Z)-6-(tert-Butoxycarbonylamino)-14a-(cyclopropylsulfonylcarbamoyl)-5,16-dioxo-1,2,3,5,6,7,8,9,10,11,13a,14,14a,15,16,16a-hexadecahydrocyclopropa[e]pyrrolo[1,2-a][1,4]diazacyclopentadecin-2-yl 1,1,3,3-d₄-4-fluoroisoindoline-2-carboxylate (111)

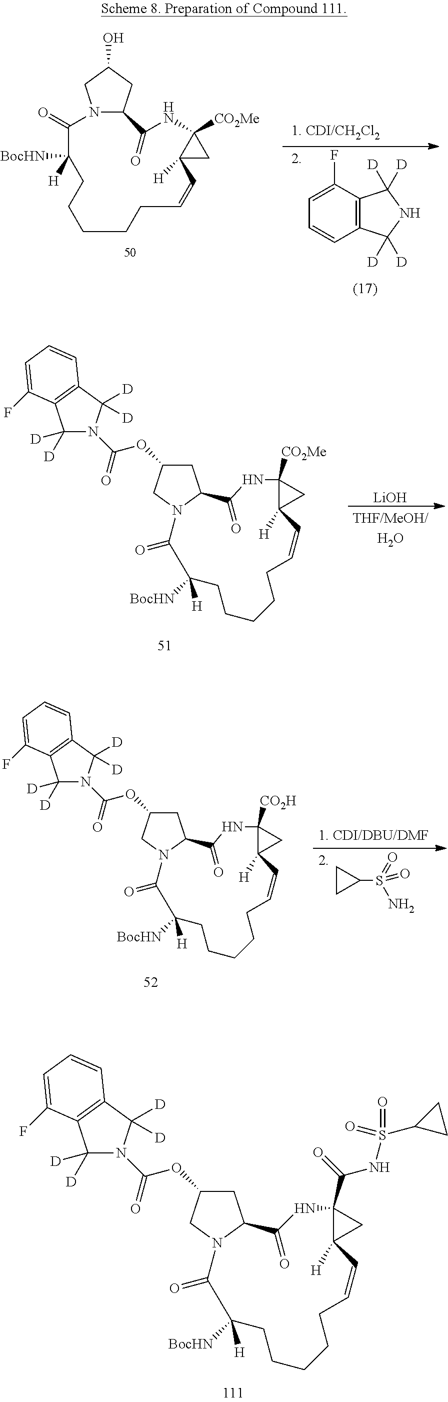

Step 1. Synthesis of (2R,6S,13aS,14aR,16aS,Z)-Methyl 6-(tert-Butoxycarbonylamino)-5,16-dioxo-2-(1,1,3,3-d₄-4-fluoroisoindoline-2-carbonyloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxylate (51). A solution of methyl ester 50 (83.0 mg, 0.173 mmol, synthesized by methods described in WO2005/037214 A2 for the preparation of the corresponding ethyl ester of compound 50) and carbonyl diimidazole (CDI) (34.0 mg, 0.208 mmol) in CH₂Cl₂ (2.00 mL) was stirred at room temperature for 15 hours. A solution of 17 (122 mg, 0.865 mmol) in 500 μL of CH₂Cl₂ was then added and the reaction was stirred for an additional 15 hours. The reaction was then diluted with CH₂Cl₂, washed successively with 1M HCl, saturated NaHCO₃, and brine, dried (MgSO₄), filtered and concentrated. The resulting residue was then purified by column chromatography (SiO₂, 0-5% MeOH/CH₂Cl₂) to afford 51 (85.0 mg, 76% yield). MS (M+H): 647.3.

Step 2. Synthesis of (2R,6S,13aS,14aR,16aS,Z)-6-(tert-butoxycarbonylamino)-5,16-dioxo-2-(1,1,3,3-d₄-4-fluoroisoindoline-2-carbonyloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxylic acid (52). A solution of 51 (68.0 mg, 0.105 mmol) and LiOH (15.0 mg, 0.631 mmol) in THF (0.500 mL), MeOH (0.250 mL) and water (0.250 mL) was stirred at room temperature for 15 hours. The reaction was then concentrated to remove THF and MeOH, diluted with 1M HCl, and extracted with CH₂Cl₂ (3×10 mL). The combined organic layers were dried (Na₂SO₄), filtered and concentrated to afford 52 (45.0 mg, 68% yield). MS (M+H): 633.3.

Step 3. Synthesis of (2R,6S,13aS,14aR,16aS,Z)-6-(tert-Butoxycarbonylamino)-14a-(cyclopropylsulfonylcarbamoyl)-5,16-dioxo-1,2,3,5,6,7,8,9,10,11,13a,14,14a,15,16,16a-hexadecahydrocyclopropa[e]pyrrolo[1,2-a][1,4]diazacyclopentadecin-2-yl 1,1,3,3-d₄-4-fluoroisoindoline-2-carboxylate (111). A solution of 52 (45 mg, 0.071 mmol) and CDI (12 mg, 0.071 mmol) in DMF (1 mL) was stirred at 40° C. for 1 hour. Cyclopropanesulfonamide (13 mg, 0.11 mmol) and DBU (11 μL, 0.071 mmol) were then added and the reaction was then stirred at 40° C. for an additional 15 hours. The reaction was then cooled to room temperature, diluted with water and extracted with ethyl acetate (3×5 mL). The combined organic layers were then washed with 1M HCl, saturated NaHCO₃, dried (Na₂SO₄), filtered and concentrated. The resulting residue was purified via chromatography (SiO₂, 0-5% MeOH/CH₂Cl₂) to afford compound 111 (12 mg, 23% yield). ¹H-NMR (400 MHz, acetone-d₆): δ 10.69 (br s, 1H), 8.42-8.24 (m, 1H), 7.40-7.31 (m, 1H), 7.19 (d, J=7.6 Hz, 1H), 7.15-7.00 (m, 2H), 6.15 (br s, 1H), 5.69 (q, J=9.3 Hz, 1H), 5.43 (br s, 1H), 5.00 (t, J=9.6 Hz, 1H), 4.64-4.56 (m, 1H), 4.51-4.43 (m, 1H), 4.19-4.05 (m, 1H), 3.89-3.79 (m, 1H), 2.86-2.78 (m, 3H), 2.71-2.60 (m, 1H), 2.52-2.35 (m, 3H), 1.92-1.81 (m, 2H), 1.74 (dd, J=5.6, 7.8 Hz, 1H), 1.62-1.06 (m, 17H), 1.03-0.91 (m, 2H). MS (M−Boc+H): 636.1.

### Example 4

### Synthesis of (2R,6S,13aS,14aR,16aS,Z)-6-((tert-Butoxy-d₉)carbonylamino)-14a-(cyclopropylsulfonylcarbamoyl)-5,16-dioxo-1,2,3,5,6,7,8,9,10,11,13a,14,14a,15,16,16a-hexadecahydrocyclopropa[e]pyrrolo[1,2-a][1,4]diazacyclopentadecin-2-yl 4-fluoroisoindoline-2-carboxylate (100)

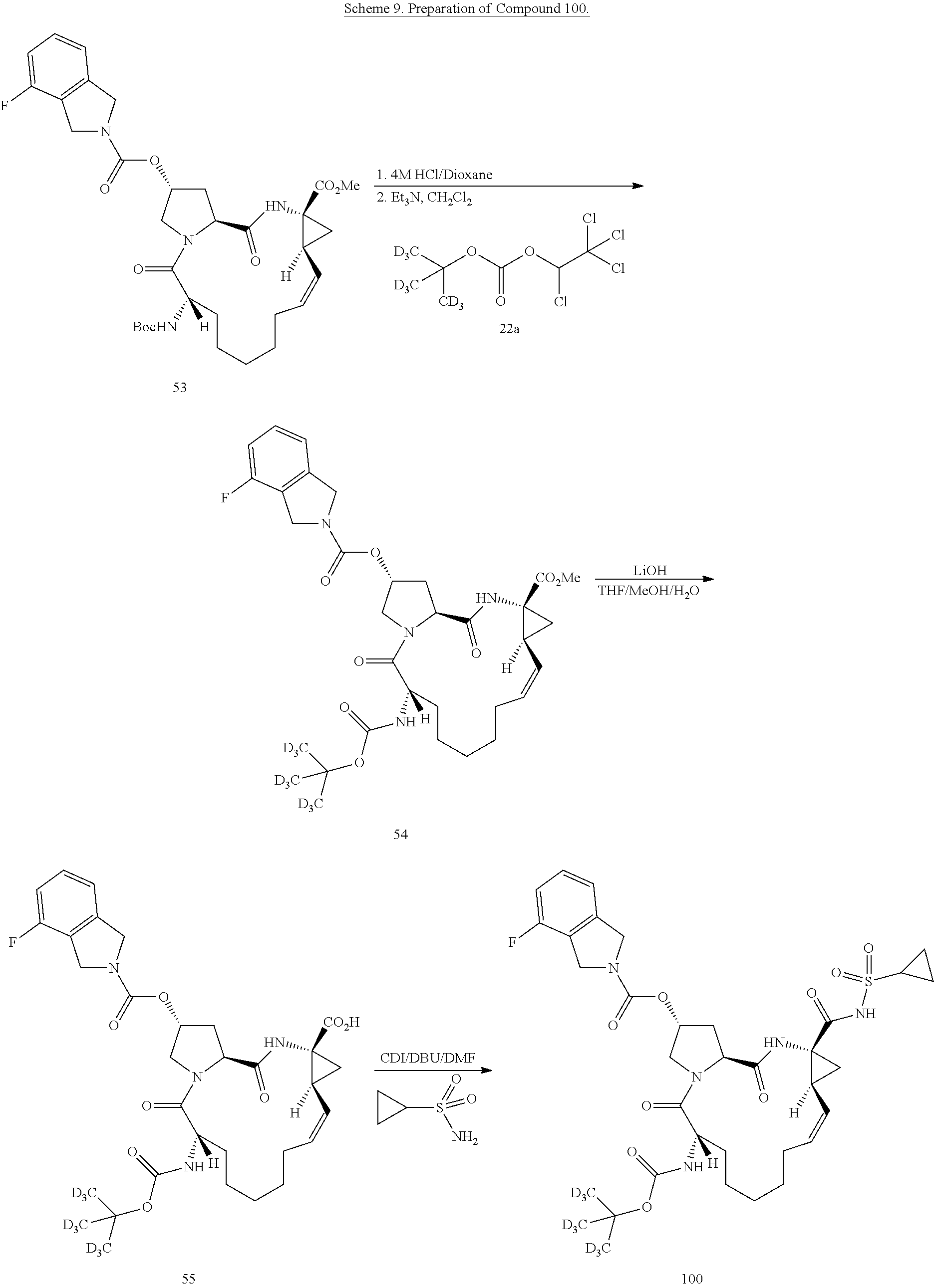

Step 1. Synthesis of (2R,6S,13aS,14aR,16aS,Z)-Methyl 6-((tert-Butoxy-d₉)carbonylamino)-5,16-dioxo-2-(4-fluoroisoindoline-2-carbonyloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxylate (54). A solution of compound 53 (82 mg, 0.13 mmole, for preparation of this compound see WO2008/086161 A1) in 4M HCl/dioxane (5.0 mL) was stirred at room temperature for 1.5 hours. The reaction was then concentrated in-vacuo to afford an off-white solid. To this solid were added CH₂Cl₂ (2.0 mL) and compound 22a (56 mg, 0.19 mmol) followed by triethylamine (53 μL, 0.38 mmol). The reaction was stirred at room temperature for 15 hours, then diluted with CH₂Cl₂ and washed with 1M HCl, saturated NaHCO₃ and brine. The organic layer was dried (MgSO₄), filtered and concentrated. The resulting residue was purified via column chromatography (SiO₂, 0-5% MeOH/CH₂Cl₂) to afford compound 54 (33 mg, 40% yield). MS (M+H): 652.5.

Step 2. Synthesis of (2R,6S,13aS,14aR,16aS,Z)-6-((tert-butoxy-d₉)carbonylamino)-5,16-dioxo-2-(4-fluoroisoindoline-2-carbonyloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxylic acid (55). A solution of compound 54 (33.0 mg, 0.052 mmol) and LiOH (7.0 mg, 0.30 mmol) in THF (0.50 mL), MeOH (0.25 mL), and water (0.25 mL) was stirred at room temperature for 15 hours. The reaction was then concentrated to remove THF and MeOH, diluted with 1M HCl, and extracted with CH₂Cl₂ (3×10 mL). The combined organic layers were dried (Na₂SO₄), filtered and concentrated to afford compound 55 (22.0 mg, 66% yield). MS (M+H): 638.3.

Step 3. Synthesis of (2R,6S,13aS,14aR,16aS,Z)-6-((tert-Butoxy-d₉)carbonylamino)-14a-(cyclopropylsulfonylcarbamoyl)-5,16-dioxo-1,2,3,5,6,7,8,9,10,11,13a,14,14a,15,16,16a-hexadecahydrocyclopropa[e]pyrrolo[1,2-a][1,4]diazacyclopentadecin-2-yl 4-fluoroisoindoline-2-carboxylate (100). A solution of compound 55 (22 mg, 0.034 mmol) and CDI (6 mg, 0.034 mmol) in DMF (0.6 mL) was stirred at 40° C. for 1 hour. Cyclopropanesulfonamide (6 mg, 0.051 mmol) and DBU (5 μL, 0.034 mmol) were then added and the reaction was stirred at 40° C. for an additional 15 hours. The reaction was cooled to room temperature, diluted with water and extracted with ethyl acetate (3×5 mL). The combined organic layers were successively washed with 1M HCl, saturated NaHCO₃, dried (Na₂SO₄), filtered and concentrated. The resulting residue was purified via column chromatography (SiO₂, 0-5% MeOH/CH₂Cl₂) to afford compound 100 (4.6 mg, 18% yield). ¹H-NMR (400 MHz, acetone-d₆) δ10.69 (br s, 1H), 8.42-8.24 (m, 1H), 7.40-7.31 (m, 1H), 7.19 (d, J=7.6 Hz, 1H), 7.15-7.00 (m, 2H), 6.15 (br s, 1H), 5.69 (q, J=9.3 Hz, 1H), 5.43 (br s, 1H), 5.00 (t, J=9.6 Hz, 1H), 4.78-4.56 (m, 5H), 4.51-4.43 (m, 1H), 4.19-4.05 (m, 1H), 3.89-3.79 (m, 1H), 2.86-2.78 (m, 3H), 2.71-2.60 (m, 1H), 2.52-2.35 (m, 3H), 1.92-1.81 (m, 2H), 1.74 (dd, J=5.6, 7.8 Hz, 1H), 1.62-1.06 (m, 8H), 1.03-0.91 (m, 2H). MS (M−1): 739.3569.

### Example 5

### Synthesis of (2R,6S,13aS,14aR,16aS,Z)-6-((tert-Butoxy-d₉)carbonylamino)-14a-(cyclopropylsulfonylcarbamoyl)-5,16-dioxo-1,2,3,5,6,7,8,9,10,11,13a,14,14a,15,16,16a-hexadecahydrocyclopropa[e]pyrrolo[1,2-a][1,4]diazacyclopentadecin-2-yl 1,1,3,3-d₄-4-fluoroisoindoline-2-carboxylate (101)

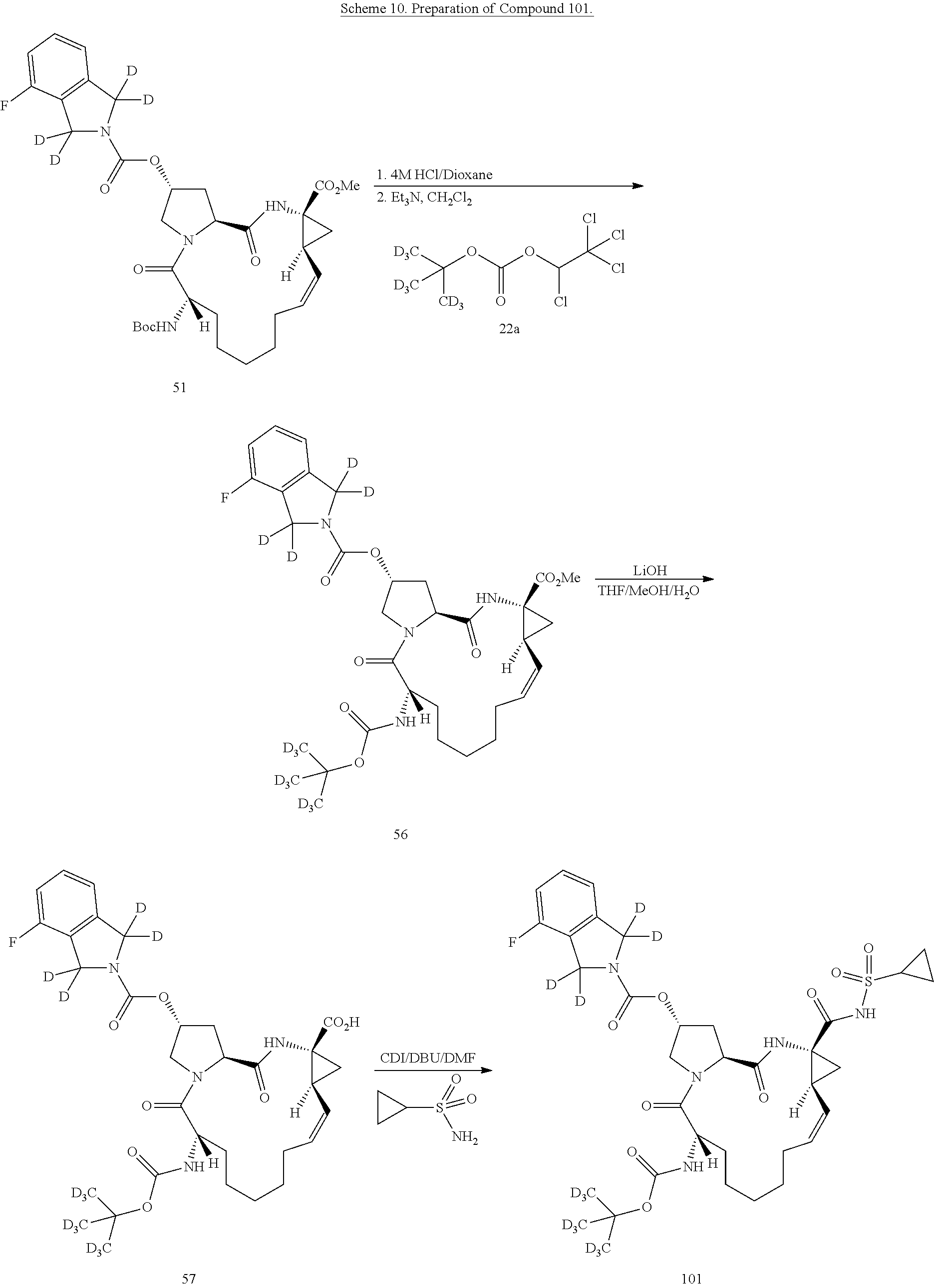

Step 1. Synthesis of (2R,6S,13aS,14aR,16aS,Z)-Methyl 6-((tert-Butoxy-d₉)carbonylamino)-5,16-dioxo-2-(1,1,3,3-d₄-4-fluoroisoindoline-2-carbonyloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxylate (56). A solution of compound 51 (85 mg, 0.13 mmol) in 4M HCl/dioxane (5.0 mL) was stirred at room temperature for 1.5 hours. The reaction mixture was then concentrated in-vacuo to afford an off-white solid. To this solid were added CH₂Cl₂ (2.0 mL) and compound 22a (58 mg, 0.20 mmol) followed by triethylamine (55 μL, 0.40 mmol). The mixture was stirred at room temperature for 15 hours, was diluted with CH₂Cl₂ and washed with 1M HCl, saturated NaHCO₃ and brine. The organic layer was then dried (MgSO₄), filtered and concentrated. The resulting residue was purified via column chromatography (SiO₂, 0-5% MeOH/CH₂Cl₂) to afford compound 56 (35 mg, 40% yield). MS (M+H): 656.4

Step 2. Synthesis of (2R,6S,13aS,14aR,16aS,Z)-6-((tert-butoxy-d₉)carbonylamino)-5,16-dioxo-2-(1,1,3,3-d₄-4-fluoroisoindoline-2-carbonyloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxylic acid (57). A solution of compound 56 (35.0 mg, 0.055 mmol) and LiOH (8.0 mg, 0.33 mmol) in THF (0.50 mL), MeOH (0.25 mL), and water (0.25 mL) was stirred at room temperature for 15 hours. The reaction mixture was concentrated to remove THF and MeOH, diluted with 1M HCl, and extracted with CH₂Cl₂ (3×10 mL). The combined organic layers were dried (Na₂SO₄), filtered and concentrated to afford compound 57 (24.0 mg, 68% yield). MS (M+H): 642.4.

Step 3. Synthesis of (2R,6S,13aS,14aR,16aS,Z)-6-((tert-Butoxy-d₉)carbonylamino)-14a-(cyclopropylsulfonylcarbamoyl)-5,16-dioxo-1,2,3,5,6,7,8,9,10,11,13a,14,14a,15,16,16a-hexadecahydrocyclopropa[e]pyrrolo[1,2-a][1,4]diazacyclopentadecin-2-yl 1,1,3,3-d₄-4-fluoroisoindoline-2-carboxylate (101). A solution of compound 57 (24 mg, 0.037 mmol) and CDI (6 mg, 0.037 mmol) in DMF (0.6 mL) was stirred at 40° C. for 1 hour. Cyclopropanesulfonamide (7 mg, 0.055 mmol) and DBU (6 μL, 0.037 mmol) were then added and the mixture was stirred for an additional 15 hours at 40° C. The mixture was cooled to room temperature, diluted with water and extracted with ethyl acetate (3×5 mL). The combined organic layers were then washed with 1M HCl, saturated NaHCO₃, dried (Na₂SO₄), filtered and concentrated. The resulting residue was purified via column chromatography (SiO₂, 0-5% MeOH/CH₂Cl₂) to afford compound 101 (9.6 mg, 35% yield). ¹H-NMR (400 MHz, acetone-d₆) δ 10.69 (br s, 1H), 8.42-8.24 (m, 1H), 7.40-7.31 (m, 1H), 7.19 (d, J=7.6 Hz, 1H), 7.15-7.00 (m, 2H), 6.15 (br s, 1H), 5.69 (q, J=9.3 Hz, 1H), 5.43 (br s, 1H), 5.00 (t, J=9.6 Hz, 1H), 4.64-4.56 (m, 1H), 4.51-4.43 (m, 1H), 4.19-4.05 (m, 1H), 3.89-3.79 (m, 1H), 2.86-2.78 (m, 3H), 2.71-2.60 (m, 1H), 2.52-2.35 (m, 3H), 1.92-1.81 (m, 2H), 1.74 (dd, J=5.6, 7.8 Hz, 1H), 1.62-1.06 (m, 8H); 1.03-0.91 (m, 2H). MS (M−1): 743.3746.

### Evaluation of Metabolic Stability

Certain in vitro liver metabolism studies have been described previously in the following references, each of which is incorporated herein in their entirety: Obach, R S, Drug Metab Disp, 1999, 27:1350; Houston, J B et al., Drug Metab Rev, 1997, 29:891; Houston, J B, Biochem Pharmacol, 1994, 47:1469; Iwatsubo, T et al., Pharmacol Ther, 1997, 73:147; and Lave, T, et al., Pharm Res, 1997, 14:152.

Microsomal Assay: Human liver microsomes (20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich. The incubation mixtures are prepared according to Table 1:

TABLE 1
Reaction Mixture Composition for Human Liver Microsome Study

| | | |
|---|---|---|
| Liver Microsomes | 3.0 | mg/mL |
| Potassium Phosphate, pH 7.4 | 100 | mM |
| Magnesium Chloride | 10 | mM |

Determination of Metabolic Stability: Two aliquots of this reaction mixture are used for a compound of this invention. The aliquots are incubated in a shaking water bath at 37° C. for 3 minutes. The test compound is then added into each aliquot at a final concentration of 0.5 μM. The reaction is initiated by the addition of cofactor (NADPH) into one aliquot (the other aliquot lacking NADPH serves as the negative control). Both aliquots are then incubated in a shaking water bath at 37° C. Fifty microliters (50 μL) of the incubation mixtures are withdrawn in triplicate from each aliquot at 0, 5, 10, 20, and 30 minutes and combined with 50 μL of ice-cold acetonitrile to terminate the reaction. The same procedure is followed for ITMN-191 and a positive control substrate for the particular metabolic enzyme being studied. Testing is done in triplicate.

Data analysis: The in vitro half-lives (t_{1/2}s) for test compounds are calculated from the slopes of the linear regression of % parent remaining (ln) vs incubation time relationship according to the following equation:

in vitro t_{1/2}=0.693/k

k=−[slope of linear regression of % parent remaining(ln) vs incubation time]

Data analysis is performed using Microsoft Excel Software.

The metabolic stability of compounds of Formula I is tested using pooled liver microsomal incubations. Full scan LC-MS analysis is then performed to detect major metabolites. Samples of the test compounds, exposed to pooled human liver microsomes, are analyzed using HPLC-MS (or MS/MS) detection. For determining metabolic stability, multiple reaction monitoring (MRM) is used to measure the disappearance of the test compounds. For metabolite detection, Q1 full scans are used as survey scans to detect the major metabolites.

SUPERSOMES™ Assay. Various human cytochrome P450-specific SUPERSOMES™ are purchased from Gentest (Woburn, Mass., USA). A 1.0 mL reaction mixture containing 25 pmole of SUPERSOMES™, 2.0 mM NADPH, 3.0 mM MgCl, and 1 μM of a compound of Formula I in 100 mM potassium phosphate buffer (pH 7.4) is incubated at 37° C. in triplicate. Positive controls contain 1 μM of ITMN-191 instead of a compound of Formula I. Negative controls use Control Insect Cell Cytosol (insect cell microsomes that lacked any human metabolic enzyme) purchased from GenTest (Woburn, Mass., USA). Aliquots (50 μL) are removed from each sample and placed in wells of a multi-well plate at various time points (e.g., 0, 2, 5, 7, 12, 20, and 30 minutes) and to each aliquot is added 50 μL of ice cold acetonitrile with 3 μM haloperidol as an internal standard to stop the reaction.

Plates containing the removed aliquots are placed in −20° C. freezer for 15 minutes to cool. After cooling, 100 μL of deionized water is added to all wells in the plate. Plates are then spun in the centrifuge for 10 minutes at 3000 rpm. A portion of the supernatant (100 μL) is then removed, placed in a new plate and analyzed using Mass Spectrometry.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

1. A compound of Formula I: 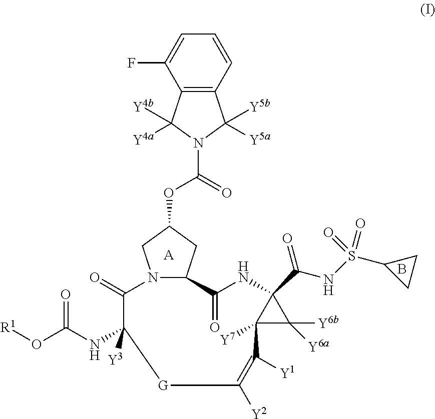
or a pharmaceutically acceptable salt thereof, wherein:
Ring A contains 0 to 6 deuterium atoms;
Ring B contains 0 to 5 deuterium atoms;
R¹ is a t-butyl group containing 0 to 9 deuterium atoms;
G is a n-pentylene group containing 0 to 10 deuterium atoms;
each Y is independently hydrogen or deuterium;
with the proviso that when Ring A contains 0 deuterium atoms and R¹ and G
each contain 0 deuterium atoms, then Ring B contains 1-5 deuterium atoms.

2. The compound of claim 1, wherein:
Ring A contains 0 or 6 deuterium atoms;
Ring B contains 0 or 5 deuterium atoms.
each Y⁴ is the same; and
each Y⁵ is the same.

3. The compound of claim 2, wherein each of Y^{4a}, Y^{4b}, Y^{5a} and Y^{5b} is the same.

4. The compound of claim 1, wherein each Y⁶ is the same.

5. The compound of claim 4, wherein each Y⁶ is hydrogen.

6. The compound of claim 5, wherein Y¹ and Y⁷ are the same.

7. The compound of claim 6, wherein Y¹ and Y⁷ are hydrogen.

8. The compound of claim 7, wherein R¹ is selected from —C(CH₃)₃ and —C(CD₃)₃.

9. The compound of claim 8, wherein each carbon atom in G is independently bound to two hydrogen atoms or two deuterium atoms.

10. The compound of claim 9, wherein G is selected from —(CH₂)₅— and —(CD₂)₅—.

11. The compound of claim 1, wherein:
G is selected from —(CH₂)₅— and —(CD₂)₅—;
each of Y^{4a}, Y^{4b}, Y^{5a} and Y^{5b} are the same;
Y¹, Y^{6a}, Y^{6b} and Y⁷ are hydrogen;
Ring A contains 0 or 6 deuterium atoms;
Ring B contains 0 or 5 deuterium atoms; and
R¹ is selected from —C(CH₃)₃ and —C(CD₃)₃.

12. The compound of claim 11, wherein the compound is:

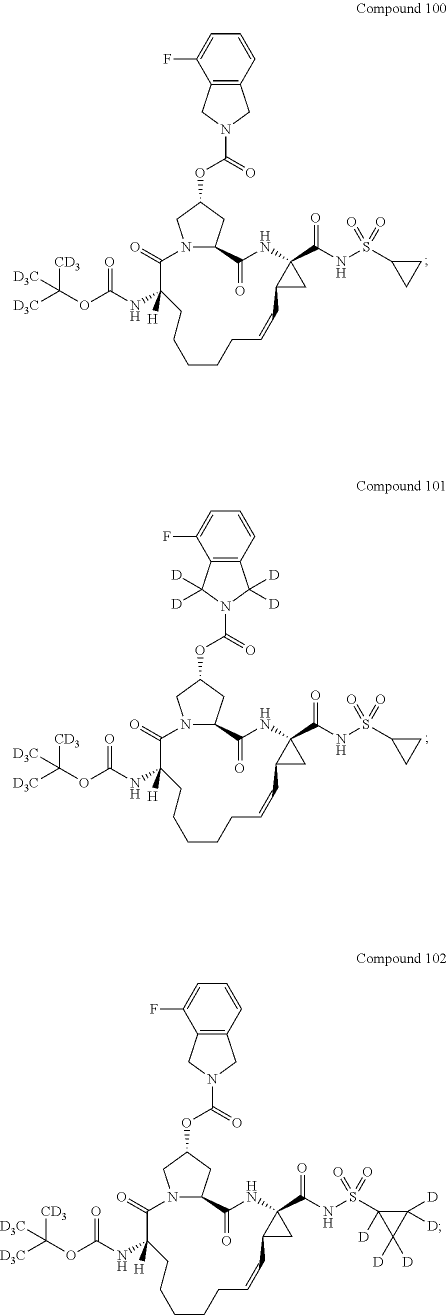

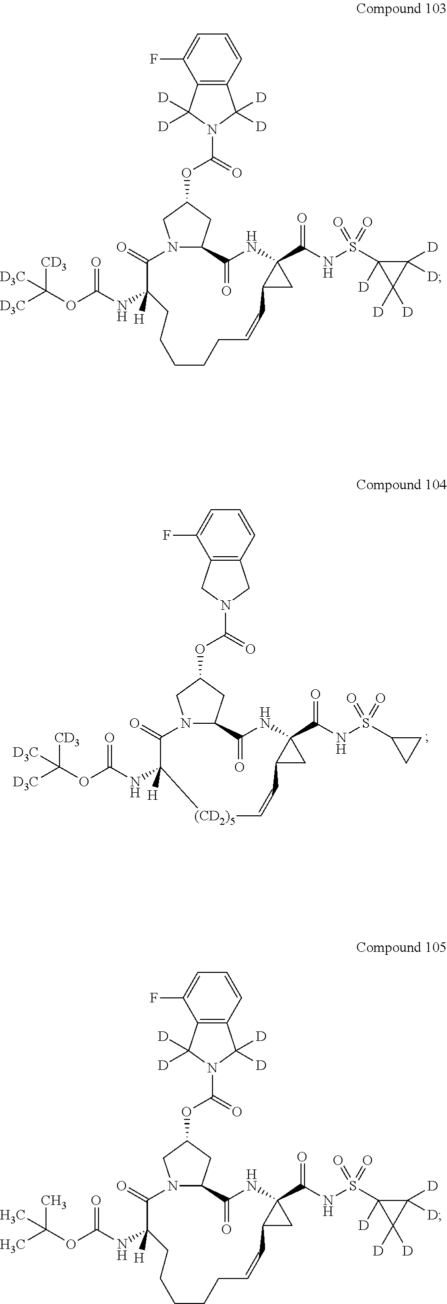

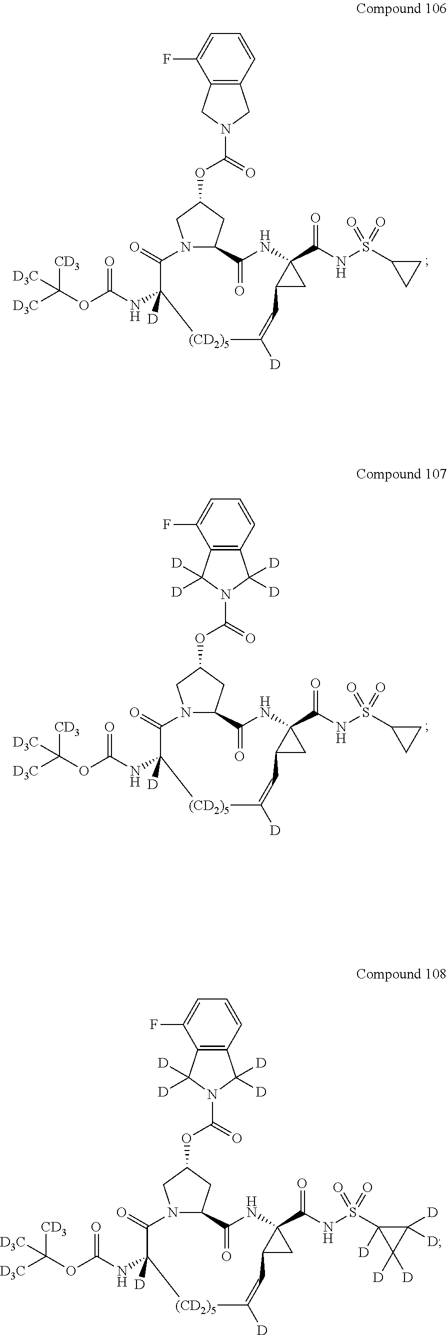

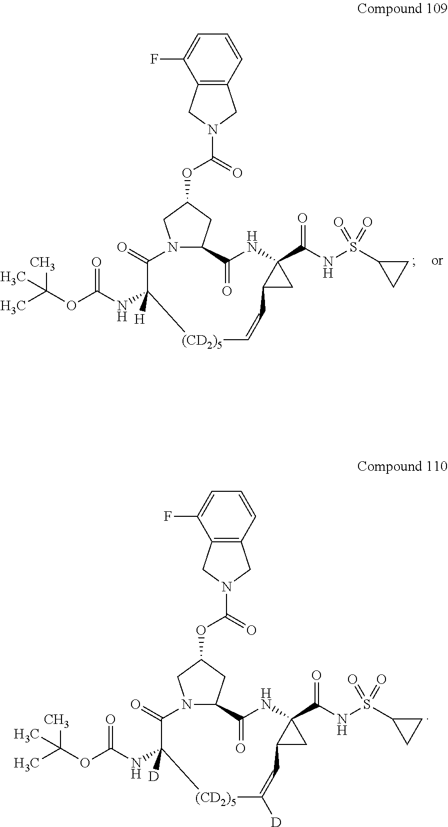

13. The compound of claim 11, wherein the compound is: 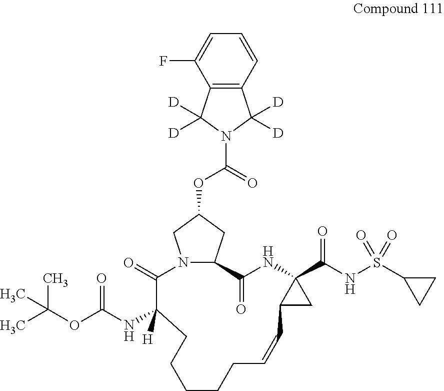
or a pharmaceutically acceptable salt thereof.

14. The compound of claim 1, wherein any atom not designated as deuterium in any of the embodiments set forth above is present at its natural isotopic abundance.

15. A pyrogen-free pharmaceutical composition comprising an effective amount of at least one compound of claim 1; and a pharmaceutically acceptable carrier.

16. (canceled)

17. (canceled)

18. A method of inhibiting the activity of a NS3 viral protease in a cell infected with a flavivirus comprising the step of contacting the cell with a compound of claim 1.

19. A method of treating a flavivirus infection or liver fibrosis in a patient in need thereof comprising the step of administering to the patient a composition of claim 15.

20. The method of claim 19, wherein the flavivirus infection is an HCV infection.

21. (canceled)

22. (canceled)
